(19) United States
(12) Patent Application Publication
(10) Pub. No.: US 2024/0358787 A1
(43) Pub. Date: Oct. 31, 2024

(54) USE OF MANGOSTEEN FRUIT SHELL EXTRACT IN THE PREPARATION OF A MEDICAMENT FOR PROMOTING WOUND HEALING IN DIABETES

(71) Applicant: Xantho Biotechnology Co., LTD, Taipei City (TW)

(72) Inventors: Ku-Cheng CHEN, New Taipei City (TW); Yen-Ju CHEN, New Taipei City (TW); Shih-Yin CHEN, New Taipei City (TW); I-Pin CHUANG, New Taipei City (TW)

(73) Assignee: Xantho Biotechnology Co., LTD, Taipei City, TW (TW)

(21) Appl. No.: 18/685,920

(22) PCT Filed: Feb. 18, 2022
(86) PCT No.: PCT/CN2022/076906

§ 371 Date: Feb. 23, 2024

(51) Int. Cl. A61K 36/38; A61P 17/02

(52) U.S. Cl. CPC A61K 36/38; A61P 17/02; A61K 2236/17; A61K 2236/331; A61K 2236/333

(57) ABSTRACT

A use of a composition in preparation of a medicament for promoting wound healing in diabetes is provided, wherein the composition comprises an effective amount of mangosteen fruit shell extract.

### CROSS-REFERENCE TO RELATED APPLICATION

This application is a national stage application, filed under 35 U.S.C. § 371, of International Patent Application No. PCT/CN2022/076906, filed on Feb. 18, 2022, which is incorporated by reference herein in its entirety.

### TECHNICAL FIELD

The present invention relates to a use of Mangosteen fruit shell extract in the preparation of a medicament for promoting wound healing in diabetes.

### BACKGROUND ART

Skin is the largest organ of the human body. There are many types of skin diseases. Skin diseases may be acute (lasting only a few minutes to several hours) or chronic conditions, which may affect individuals for days, months, years and even the entire life. Skin diseases may be conditions caused by fungal, bacterial, or viral sources, or may be non-infectious, immune responses, such as inflammatory reactions with or without allergens, or idiopathic. Therefore, the symptoms of the skin diseases may vary and range from mild itching, redness and swelling to severe pus and nociceptive pain, for examples damaging ulceration. Skin diseases may impose significant impact on the quality of an individual's life.

Diabetes mellitus (DM) is a metabolic disease since the impairment of insulin production and/or function and leads to hyperglycemia. DM patients are usually accompanied by many complications, one of them is the impairment of self-repairing abilities.

The impairment of wound healing in DM is related to several factors including vascular, neuropathic, immune, and biochemical components, and all of them are caused by hyperglycemia. Hyperglycemia leads vascular sclerosis which cause slower circulation and microvascular dysfunction, causing reduced tissue oxygenation. Hyperglycemia also reduces leukocyte migration into the wound, which becomes more vulnerable to infections, and peripheral neuropathy in DM can lead to numbness of the area and reduced ability to feel pain, which can lead to chronicization of wounds that are not immediately noticed and properly treated (Spampinato S F, Caruso G I, De Pasquale R, Sortino M A, Merlo S. The Treatment of Impaired Wound Healing in Diabetes: Looking among Old Drugs. Pharmaceuticals (Basel). 2020; 13 (4): 60.).

Particularly, diabetic foot ulcer (DFU) is a major complication of DM, which occurs in 15% of people with diabetes. The risk factors implicated in DFU are considered to be neuropathy, vascular disease, and infection. Studies have indicated that the risk of lower extremity amputation is 15 to 46 times higher in DM than in persons who do not have DM.

Clinical treatment of DFU should include blood sugar control, relief of plantar pressure, antibiotic, improvement of peripheral blood circulation, wound dressings, and surgery.

Mangosteen has been used in the field of breast cancer prevention and muscle-related diseases, it has also been developed as nutritional supplements and cosmetics in daily lives, as well as uses in the treatment of acute hepatitis, liver fibrosis and cirrhosis prevention.

Matsumoto et al., have studied α-mangostin, β-mangostin, γ-mangostin, and methyl-β-mangostin purified from Mangosteen fruit shells and investigated the inhibitory effect of this compound at various stages of the cell cycle, showing that this compound has anti-cell proliferative effect and anti-tumor effect (Bioorg. Med. Chem. 2005, 13, 6064-6069).

### SUMMARY OF THE INVENTION

The present invention provides a method for promoting wound healing in diabetes in a subject, comprising administering a pharmaceutical composition comprises an effective amount of mangosteen fruit shell extract.

In a preferred embodiment, the Mangosteen fruit shell is water extract of Mangosteen fruit shell and/or alcohol extract of Mangosteen fruit shell.

In another preferred embodiment, the Mangosteen fruit shell is the inner shell of the Mangosteen fruit shell.

### DETAILED DESCRIPTION OF THE INVENTION

The present invention provides a use of a composition in preparation of a pharmaceutical composition for treating skin disorders.

Specifically, the present invention provides a use of a composition in preparation of a medicament for promoting wound healing in diabetes, wherein the composition comprises an effective amount of extract of Mangosteen fruit shell. The medicament can also be used for topical treatment use, medical device or for precision treatment use.

The present invention provides a method for promoting wound healing in diabetes in a subject, comprising administering a pharmaceutical composition comprises an effective amount of mangosteen fruit shell extract to the subject in need thereof.

Mangosteen fruit shell contains a softer inner shell and a harder outer shell.

In a preferred embodiment, the Mangosteen fruit shell is extracted with a solvent which is selected from the group consisting of methanol, ethanol, n-propanol, 2-propanol, n-butanol, acetone, ethyl acetate and water.

In another preferred embodiment, the extract of Mangosteen fruit shell is water extract of Mangosteen fruit shell and/or alcohol extract of Mangosteen fruit shell.

In a preferred embodiment, the extract of Mangosteen fruit shell is a Mangosteen fruit shell water extract.

In another preferred embodiment, the extract of Mangosteen fruit shell is Mangosteen fruit shell alcohol extract.

In a preferred embodiment, the Mangosteen fruit shell is the inner shell/outer shell of the Mangosteen fruit shell and/or the whole shell of the Mangosteen fruit shell.

In another preferred embodiment, the Mangosteen fruit shell is the outer shell of the Mangosteen fruit shell.

In a preferred embodiment, the compositions of the present invention can be oral or parenteral preparations, the parenteral preparations can be external preparations which can be creams, pastes, ointments, gels, wash lotions or patches.

In a preferred embodiment, the extract of Mangosteen fruit shell of the present invention comprises α-mangostin and γ-mangostin.

In another preferred embodiment, the water extract of Mangosteen fruit shell of the present invention comprises α-mangostin and γ-mangostin.

In yet another preferred embodiment, the alcohol extract of Mangosteen fruit shell of the present invention comprises α-mangostin and γ-mangostin.

As used herein, the term “Effective amount” is the amount that can achieve effective results when administered to an individual, or that has the desired activity in vivo or in vitro. In the case of delayed wound healing in DM, as compared to no treatment, effective clinical outcomes include amelioration of the extent or severity of the symptoms associated with the disease or condition, and/or prolonging the life of an individual and/or improvement of the quality of life of the individual. The exact amount of compound administered to an individual will depend on the type and severity of the disease or symptoms and on the individual characteristics such as the general health of the individual, age, sex, weight, and drug tolerance of the individual. It is also dictated by the conditions, severity and types of the inflammatory disorder, the autoimmune disorder and the allergic disorder, or the desired immunosuppressive effect. Those skilled in the art will be able to determine the appropriate dose based on these and other factors.

In an embodiment, the effective amount of extract of Mangosteen fruit shell is 0.5% (w/w) to 20% (w/w). In a preferred embodiment, the effective amount of extract of Mangosteen fruit shell is 1% (w/w) to 15% (w/w). In a most preferred embodiment, the effective amount of extract of Mangosteen fruit shell is 1.25% (w/w) to 10% (w/w).

The pharmaceutical composition of the present invention can be formulated into various forms of oral or parenteral preparations. Oral preparations can be formulated as solid preparations such as powders, granules, troches, capsules, etc., or formulated as liquid preparations such as suspensions, emulsions, syrups, etc. Parenteral preparations can be formulated as external preparations such as creams, ointments, gels, wash lotions, patches, etc., or as inhalants, aerosols, suppositories, etc.

The pharmaceutical composition of the present invention can comprise pharmaceutically acceptable excipients, especially can further comprise predetermined solvents or oils, PH adjuster and if desired, can further comprise a dispersant.

Examples of solvents used in the present invention include, but are not limited to, water, ethanol, isopropanol, 1,3-butanediol, propylene glycol, glycerin, etc.

Examples of oils used in the present invention are selected from the group consisting of, but are not limited to, corn oil, sesame oil, flaxseed oil, cottonseed oil, soybean oil, peanut oil, mono-glycerides, di-glycerides, tri-glycerides, mineral oil, squalene, jojoba oil, olive oil, evening primrose oil, borage oil, grape seed oil, coconut oil, sunflower oil, shea butter, and any combinations thereof.

Solvents and oils can be used alone or in any combinations thereof.

Examples of useful dispersants include, but are not limited to, lecithin, organic monoglycerides, sorbitan fatty acid esters, polyoxyethylene fatty acid esters, sorbitan stearate, etc. These raw materials can also be used alone or in any combinations thereof.

If desired, the composition further comprises additional materials such as antimicrobials or preservatives.

In the meantime, it is known that an active ingredient can be used simultaneously with the composition as long as it does not have any adverse effects on the pharmaceutical activity of the composition of the present invention. For example, ceramide moisturizers are commonly used as conventional agents for atopic dermatitis, or liquid ingredients such as hydrocortisone steroids, vitamin A derivatives such as vitamin A palmitate and/or tocopherol, etc., can be used with the composition.

When the pharmaceutical composition is used as an external preparation, an appropriate external skin preparation can be used as a base material, and an aqueous solution, a non-aqueous solvent, a suspension, an emulsion or a lyophilized preparation, etc., can be used and sterilized according to known methods.

In practical use of the provided or administered composition of the present invention, the dosage can be determined depending on various factors such as the route of administration, the age, sex, and weight of the patient, the severity of the disease, and the type of medicament as the active ingredient.

In the case where the composition of the present invention can be a food or a cosmetic composition, the composition can be prepared by appropriate addition of at least one food supplement or a cosmetically acceptable carrier.

The food composition can be used in or added to, for example, healthy foods. As used herein, the term “healthy food” refers to a food product containing the composition of the present invention that has an enhanced function as compared to general food products. Healthy foods can be prepared by adding a general food to the composition or by encapsulation, pulverization or suspension liquefaction.

The cosmetic composition can be added alone or together with other cosmetic ingredients or can be appropriately used according to other known methods. Cosmetics include, but are not limited to, aftershaves, lotions, creams, facial masks, and color makeups.

Cosmetic compositions can be formulated into various forms of compositions, such as gels, creams, ointments, etc. The compositions in the form of gels, creams and ointments can be appropriately prepared according to the form of the composition by using known methods, and by addition of known softeners, emulsifiers and thickeners or other materials known in the art.

The gel-form composition can be prepared, for example, by addition of a softener such as trimethylolpropane, polyethylene glycol and glycerol, for example, a solvent of propylene glycol, ethanol and isocetyl alcohol, and pure water.

The preparation of the compositions in the form of creams can be carried out, for example, by addition of fatty alcohols such as stearyl alcohol, myristyl alcohol, behenyl alcohol, resveratrol, isostearyl alcohol and isocetyl alcohol; emulsifiers such lipids, such as as lecithin, phosphatidylcholine, phosphatidylethanolamine, phosphatidyl serine, phosphoinositide and derivatives thereof, glyceryl stearate, sorbitol palmitate, sorbitol stearate, etc; natural fats And oils such as avocado oil, almond oil, babassu oil, borage oil, camellia oil, etc; lipid compositions such as ceramides, cholesterol, fatty acids, phytosphingosine, lecithin, etc; solvents, such as propylene glycol, etc; and pure water.

The preparation of the compositions in the form of ointments can be carried out, for example, by addition of emollients, emulsifiers and waxes, for example microcrystalline wax, paraffin, ceresin, beeswax, spermaceti, petrolatum, etc.

In another aspect, the present invention provides a method for using the composition to prepare a medicament for treating or alleviating delayed wound healing in DM. As used herein, the term “treating or alleviating” means that when a patient uses a medicament, it stops or delays the course or symptoms of the disease.

### BRIEF DESCRIPTION OF THE DRAWINGS

FIG. 1 shows the wound healing efficacy of each test articles compared with vehicle control (disease control) and normal control in the invention. FIG. 1A: 1.25% TA1; FIG. 1B: 2.5% TA1; FIG. 1C: 5% TA1; FIG. 1D: 2.5% TA2; FIG. 1E: 5% TA2; FIG. 1F: 2.5% TA3; FIG. 1G: 5% TA3.

FIG. 2 shows the wound healing efficacy of 10% TA1 compared with vehicle control (disease control) and normal control in the invention.

### EMBODIMENTS

The examples below are non-limiting and are merely representative of various aspects and features of the present invention.

### Examples

### Materials

### Test Articles (TA)

Mangosteen fruit shell was collected and dried to 50% to 95%, extracted with a solvent (such as water or 10% to 95% alcohol), and concentrated to obtain an extract of mangosteen fruit shell.

The outer shell and inner shell of the mangosteen fruit shell were separated, the outer shell of the mangosteen fruit shell and the inner shell of the mangosteen fruit shell were respectively dried to 50% to 95% and extracted with a solvent (such as water or 10% to 95% alcohol), then concentrated to obtain an extract of mangosteen outer shell and an extract of mangosteen inner shell.

Different concentrations of pastes or ointments were prepared from the mangosteen fruit whole shell alcohol and water extract (labeled test article 1; TA1), the mangosteen fruit outer shell (labeled test article 2; TA2), and mangosteen fruit inner shell (labeled test article 3; TA3) alcohol and water extract.

### Animals

Adult (7-8 weeks old) male Crl: CDR (SD) rats with average body weight 200-250 g were used. Ear-notch and cage tag were used for animal identification. Animals were housed individually in a polycarbonate cage with the following conditions: temperature was set to maintain at 22±3° C.; humidity was set to maintain at 50±20%; 12-hr/12-hr light/dark cycle. Food and water were supplied ad libitum throughout the study period.

### Methods

### Streptozotocin (STZ)-Induced Diabetic Models

Total eighty-four rats were used for this invention. Twelve rats were used as normal group, and the remained seventy-two rats were used for induction of Type I diabetic by single dose (50 mg/kg) intraperitoneal (IP) injection of streptozotocin (STZ; Sigma Aldrich S0130). The concentration of 5 mg/mL STZ was freshly prepared in 0.1 M citrate buffer (pH=4˜4.5).

The fasting blood glucose level was measured for each STZ-treated rat using a glucometer in weeks 2, 4, 6 and 7 after STZ administration. Diagnostic criteria for diabetic in this model included: blood glucose level >250 mg/dL, weight loss, polyuria, and polydipsia.

A week after diabetic was confirmed, all diabetic rats were weighed, and fasting blood glucose concentration was measured. Any rat showing blood glucose concentration <250 mg/dL or increase in body weight was removed from the study.

Fifty-four rats were allocated into nine groups (6 rats per group) for this study. Group number, treatment, test article, and numbers of animal for each group are presented as table below:

| Group | | | Number of |
|---|---|---|---|
| No. | Treatment | Test Articles | Animals |
| 1 | Wound + Vehicle | Vehicle | 6 rats |
| | | (Normal Control) | |
| 2 | STZ + Wound + Vehicle | Vehicle | 6 rats |
| | | (Disease Control) | |
| 3 | STZ + Wound + 1.25% TA1 | TA1 | 6 rats |
| 4 | STZ + Wound + 2.5% TA1 | TA1 | 6 rats |
| 5 | STZ + Wound + 5% TA1 | TA1 | 6 rats |
| 6 | STZ + Wound + 2.5% TA2 | TA2 | 6 rats |
| 7 | STZ + Wound + 5% TA2 | TA2 | 6 rats |
| 8 | STZ + Wound + 2.5% TA3 | TA3 | 6 rats |
| 9 | STZ + Wound + 5% TA3 | TA3 | 6 rats |

The wound healing efficacy of higher dosage test articles were further evaluated. 10% test article 1, test article 2 and test article 3 were administered to rats, total thirty male rats (6 rats per group) were used for the evaluation. The same operations were performed for this study. Group number, treatment, test article, and numbers of animal for each group are presented as table below:

| Group | | | Number of |
|---|---|---|---|
| No. | Treatment | Test Articles | Animals |
| 10 | Wound + Vehicle | Vehicle | 6 rats |
| | | (Normal Control) | |
| 11 | STZ + Wound + Vehicle | Vehicle | 6 rats |
| | | (Disease Control) | |
| 12 | STZ + Wound + 10% TA1 | TA1 | 6 rats |
| 13 | STZ + Wound + 10% TA2 | TA2 | 6 rats |
| 14 | STZ + Wound + 10% TA3 | TA3 | 6 rats |

### Establishment of Wound Models

STZ-induced diabetic animal or heath animal was anesthetized, the back of all rats was shaved, and a full-thickness excisional wound (20 mm×20 mm) was made to the level of the panniculus carnosus muscle. Wounds were not sutured or covered but were allowed to be healed by secondary intention. Wounds were inflicted in the same manner for all study rats.

### Topical Application

Each Test articles (whole shell extract, inner shell extract, and outer shell extract) or vehicle control formulation was applied topically in approximately 0.25 mm thick film (total 0.1 g/wound) to cover the entire wound once a day. Wounds were cleaned gently with saline prior to each application; total of twenty-one applications were performed for each rat.

Animal observation was performed for all study animals at least once daily, body weights were recorded on Day 0 (prior to dosing), at least weekly and the day of necropsy for all surviving animals.

### Wound Measurement

Gross observations were made on each wound daily during topical application period. Wound area was measured in two different ways: (i) tracing onto clear plastic sheets once every 3 days, and (ii) recording digital images using digital camera once every 3 days.

Wound area was calculated using Image J. The initial (Day 0) wound area following the creation of wound was used to calculate % wound closure for each wound on any given day. The percentage of wound area covered by new granulation tissue={[(Areaᵢ(Aᵢ)−Areaₙ (Aₙ)]/Areaᵢ (Aᵢ)}×100, where Aᵢ is the initial area and Aₙ is the area at day n.

### Analysis

Statistical analysis was performed using SigmaPlot™ Statistical Software for Windows™, Release 12.0 (Jandel Scientific Inc., USA). A significance level of 0.05 is used for all statistical tests.

### Result

### Diabetic Model

All rats used in the present study showed the characteristic signs of hyperglycemia from the second day after the administration of streptozotocin (STZ). Blood glucose levels increased significantly in all groups after 48 hours of STZ administration and remained elevated throughout the experiment (Table 1). At animal termination (Day 21), all animals were under the status of diabetic except group 1.

TABLE 1

Fasting Blood Glucose (mg/dL)

| Group | Treatment | Prior to Dosing | Termination |
|---|---|---|---|
| 1 | Wound + Vehicle | — | 100.33 ± 17.53* |
| 2 | STZ + Wound + Vehicle | 410.00 ± 82.72 | 286.67 ± 144.36 |
| 3 | STZ + Wound + 1.25% TA1 | 441.67 ± 57.53 | 356.80 ± 117.26 |
| 4 | STZ + Wound + 2.5% TA1 | 454.83 ± 89.24 | 359.67 ± 146.43 |
| 5 | STZ + Wound + 5% TA1 | 458.00 ± 66.36 | 306.00 ± 108.38 |
| 6 | STZ + Wound + 2.5% TA2 | 441.83 ± 35.39 | 404.50 ± 101.38 |
| 7 | STZ + Wound + 5% TA2 | 458.00 ± 59.78 | 346.33 ± 125.57 |
| 8 | STZ + Wound + 2.5% TA3 | 457.50 ± 65.42 | 396.83 ± 134.38 |
| 9 | STZ + Wound + 5% TA3 | 449.67 ± 44.14 | 364.17 ± 124.28 |

*P < 0.05 (Compared to Group 2)

### Wound Healing

The following results were obtained during study of wound healing in control and diabetic experimental rats (Table 2).

TABLE 2

| | Group | | | | |
|---|---|---|---|---|---|
| | 1 | 2 | 3 | 4 | 5 |
| | Treatment | | | | |
| | | STZ + | STZ + | STZ + | STZ + |
| | Wound + | Wound + | Wound + | Wound + | Wound + |
| | Vehicle | Vehicle | 1.25% TA1 | 2.5% TA1 | 5% TA1 |
| Day 0 | 0 | 0 | 0 | 0 | 0 |
| Day 3 | 8.42 ± 13.35* | −22.48 ± 14.85 | −10.51 ± 18.61 | −12.78 ± 10.04 | −5.27 ± 19.57 |
| Day 6 | 18.43 ± 12.65* | −21.48 ± 10.07 | −14.00 ± 16.63 | −7.23 ± 13.10 | −0.64 ± 10.05 |
| Day 9 | 55.63 ± 8.78* | 3.54 ± 14.12 | 1.95 ± 30.13 | 13.08 ± 14.38 | 20.16 ± 8.43 |
| Day 12 | 81.62 ± 4.59* | 24.05 ± 13.09 | 39.07 ± 19.80 | 44.17 ± 19.03 | 50.13 ± 9.27 |
| Day 15 | 89.15 ± 4.30* | 39.87 ± 7.32 | 63.05 ± 13.69* | 60.48 ± 16.04* | 64.20 ± 8.53* |
| Day 18 | 94.29 ± 2.12* | 54.37 ± 8.13 | 73.21 ± 10.31 | 72.81 ± 13.47 | 78.26 ± 2.91* |
| Day 21 | 96.30 ± 2.56* | 63.18 ± 10.65 | 85.61 ± 4.64 | 86.42 ± 4.72 | 88.35 ± 2.68* |

Group

| | | | |
|---|---|---|---|
| 6 | 7 | 8 | 9 |

Treatment

| | STZ + | STZ + | STZ + | STZ + |
|---|---|---|---|---|
| | Wound + | Wound + | Wound + | Wound + |
| | 2.5% TA2 | 5% TA2 | 2.5% TA3 | 5% TA3 |
| Day 0 | 0 | 0 | 0 | 0 |
| Day 3 | −17.68 ± 11.38 | −14.48 ± 18.65 | −8.34 ± 19.84 | −13.58 ± 7.19 |
| Day 6 | −10.13 ± 11.69 | −1.73 ± 18.50 | 1.81 ± 12.31 | −5.72 ± 20.51 |
| Day 9 | 23.95 ± 18.48 | 15.70 ± 16.23 | 24.28 ± 10.11 | 32.44 ± 15.50* |
| Day 12 | 53.88 ± 14.42 | 48.56 ± 10.45 | 53.71 ± 11.88* | 60.84 ± 9.34* |
| Day 15 | 70.14 ± 12.07* | 67.03 ± 10.81* | 66.93 ± 15.65* | 75.71 ± 12.62* |
| Day 18 | 81.31 ± 9.90* | 80.34 ± 10.06* | 81.34 ± 11.92* | 86.23 ± 3.99* |
| Day 21 | 85.97 ± 11.46 | 84.66 ± 8.62 | 88.46 ± 10.31* | 92.87 ± 3.14* |

The wound areas measured on different days i.e. 3^{rd}, 6^{th}, 9^{th}, 12^{th}, 15^{th}, 18^{th} and 21^{st} after wound creation show that topically applied the test articles (TA1, TA2 and TA3) had a positive effect on the healing of the diabetic wound. It was observed that the rate of wound closure in disease control rats (Group 2) has decreased in comparison with the normal control animals (Group 1); this indicated chronic wound healing model used in this study was verified. This effect was obvious from 3rd day onward.

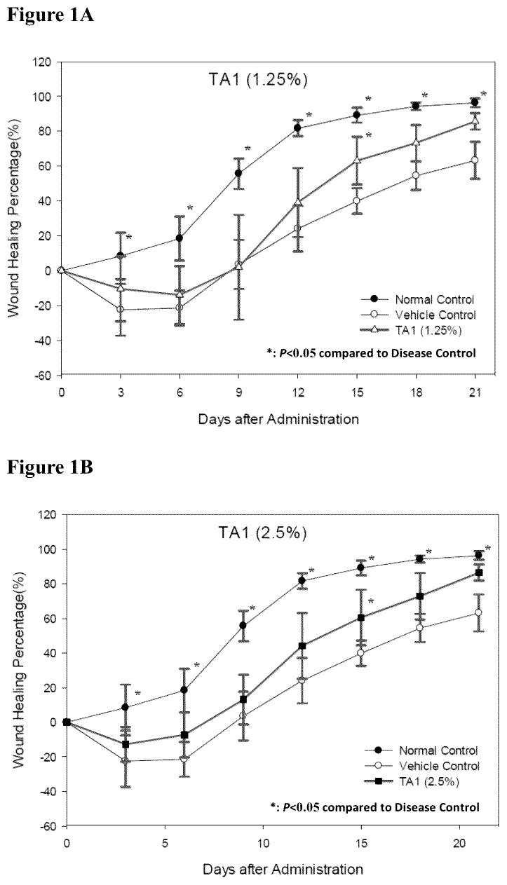

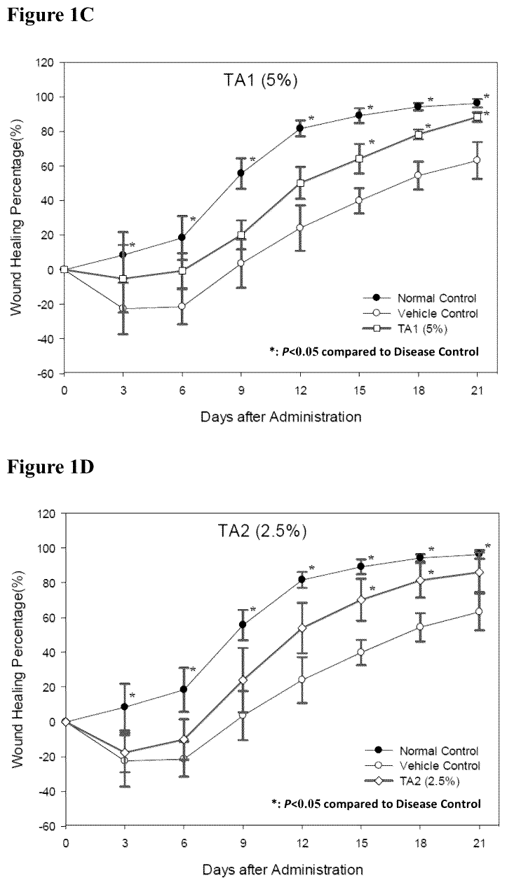

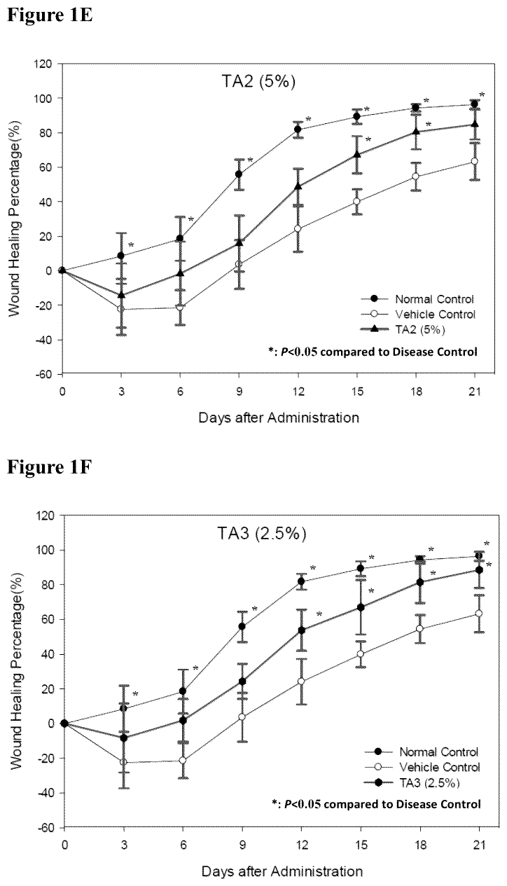

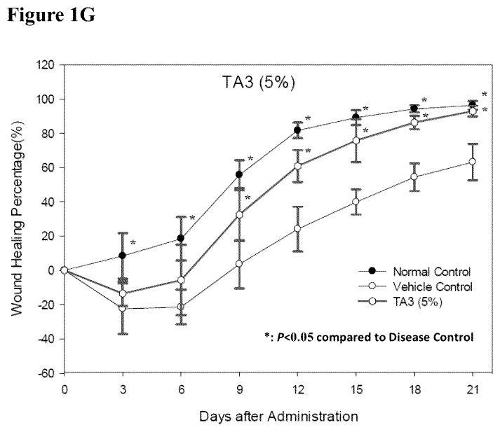

As shown in FIG. 1, the wound contracting ability of experimental rats receiving TA1 topical administration (Group 3: FIG. 1A, Group 4: FIG. 1B, and Group 5: FIG. 1C) showed significant wound healing when compared with disease control rats (Group 2) from day 15. The maximum percentages (rate) of wound closure were observed in 5% TA1 treated animals (Group 5) compared to disease control rats (Group 2) at day 21: the percent wound closures were 88.35±2.68 (P<0.05) for 5% TA1 and 63.18±10.65 for Group 2.

The wound contracting ability of experimental rats receiving TA2 topical administration (Group 6: FIG. 1D, and Group 7: FIG. 1E) showed significant wound healing from the 15^{th} day onward when compared with disease control rats (Group 2).

The wound contracting ability of experimental rats receiving TA3 topical administration (Group 8: FIG. 1F and Group 9: FIG. 1G) showed significant wound healing from the 15^{th} day onward when compared with disease control rats (Group 2).

In summary, based on the microscopic findings of this study, the test articles, mangosteen fruit whole shell (TA1), mangosteen fruit outer shell (TA2) and mangosteen fruit inner shell (TA3), had the potential to enhance wound healing due to better epidermal and dermal regeneration, as well as granulation tissue formation and epidermal migration.

In the conclusion, TA1, TA2 and TA3 significantly improved delayed wound healing in hyperglycemic rats. 5% of mangosteen fruit whole shell, as well as 2.5% and 5% of mangosteen fruit inner shell had best wound healing efficacy with decreased the concentration of pro-inflammatory cytokines, these were considered to be as top formulations. Comparison of the same concentration (5%) of three test articles, the order ranking of wound healing responses were mangosteen fruit inner shell as best, mangosteen fruit whole shell as second and mangosteen fruit outer shell as third.

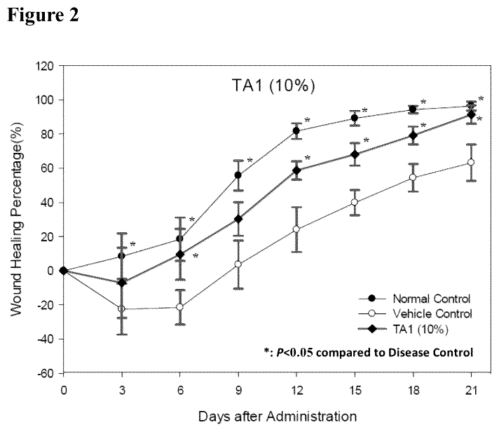

For the results of wound healing efficacy of 10% TA1, TA2 and TA3, 10% TA1 showed significant wound healing from the 12^{th} day onward when compared with disease control rats (FIG. 2), 10% TA1 showed better wound healing efficacy than 5% TA1. The TA2 and TA3 also showed same tendency, and the order ranking of wound healing responses were also mangosteen fruit inner shell as best, mangosteen fruit whole shell as second and mangosteen fruit outer shell as third.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cells, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

1. A method for promoting wound healing in diabetes, comprising administering a pharmaceutical composition comprises an effective amount of mangosteen fruit shell extract to a subject in need thereof.

2. The method of claim 1, wherein the extract of mangosteen fruit shell is water extract of mangosteen fruit shell and/or alcohol extract of mangosteen fruit shell.

3. The method of claim 1, wherein the mangosteen fruit shell is outer shell of the mangosteen fruit shell and/or inner shell of the mangosteen fruit shell.

4. The method of claim 1, wherein the mangosteen fruit shell is inner shell of the mangosteen fruit shell.

5. The method of claim 1, wherein the extract of mangosteen fruit shell comprises α-mangostin and γ-mangostin.

6. The method of claim 1, wherein the composition is a parenteral preparation.

7. The method of claim 6, wherein the parenteral preparation is an external preparation.

8. The method of claim 1, wherein the effective amount of extract of Mangosteen fruit shell is 0.5% (w/w) to 20% (w/w).

9. The method of claim 1, wherein the effective amount of extract of Mangosteen fruit shell is 1% (w/w) to 15% (w/w).

10. The method of claim 1, wherein the effective amount of extract of Mangosteen fruit shell is 1.25% (w/w) to 10% (w/w).

11. The method of claim 1, wherein the wound comprises diabetic foot ulcer (DFU).
